(19) United States
(12) Patent Application Publication
(10) Pub. No.: US 2026/0171218 A1
(43) Pub. Date: Jun. 18, 2026

(54) INFORMATION PROCESSING APPARATUS, INFORMATION PROCESSING METHOD, AND STORAGE MEDIUM

(71) Applicant: Konica Minolta, Inc., Tokyo (JP)

(72) Inventors: Kazuki SASABUCHI, Tokyo (JP); Hitoshi FUTAMURA, Tokyo (JP); Naoki HAYASHI, Tokyo (JP)

(21) Appl. No.: 19/416,423

(22) Filed: Dec. 11, 2025

(30) Foreign Application Priority Data
Dec. 16, 2024 (JP) 2024-219601

(51) Int. Cl. G16H 30/20

(52) U.S. Cl. CPC G16H 30/20

(57) ABSTRACT

An information processing apparatus that creates examination order information for capturing a medical image includes a hardware processor that acquires at least one reference image candidate, selects a reference image from the at least one reference image candidate, causes a display to display examination order information linked with the selected reference image, and outputs the reference image in association with the examination order information.

### CROSS-REFERENCE TO RELATED APPLICATION

The entire disclosure of Japanese Patent Application No. 2024-219601, filed on Dec. 16, 2024, including description, claims, drawings and abstract is incorporated herein by reference.

### BACKGROUND OF THE INVENTION

### Technical Field

The present disclosure relates to an information processing apparatus, an information processing method, and a storage medium.

### Description of Related Art

Conventionally, in order to diagnose a patient, a person in charge of an examination(s) (hereinafter “examiner”) captures a medical image on the basis of examination order information from a requesting doctor. Creation of examination order information requires a certain level of knowledge of radiography. In addition, the number of pieces of examination order information created by one requesting doctor is often huge, and creation of examination order information imposes a large load on the requesting doctor.

Japanese Unexamined Patent Publication No. 2012-108761 discloses that a past schema image is registered in advance, and examination order information linked with the schema image is called, thereby saving labor for creating examination order information.

Japanese Unexamined Patent Publication No. 2023-101391 discloses that, in order to accurately and easily convey an instruction content related to an examination, reference image candidates are acquired on the basis of input patient information, and a reference image is attached to examination order information.

### SUMMARY OF THE INVENTION

According to Japanese Unexamined Patent Publication No. 2012-108761, although labor for creating examination order information is saved, information is transmitted between the requesting doctor and the examiner by using examination order information formed of only a text, miscommunication may occur between the requesting doctor and the examiner.

It is conceivable to reduce miscommunication by attaching a reference image to examination order information as in Japanese Unexamined Patent Publication No. 2023-101391, but since the reference image is acquired based on patient information, the requesting doctor needs to input the patient information, which takes time and effort.

An object of the present disclosure is to reduce miscommunication between a requesting doctor and an examiner without troubling the requesting doctor in creation of examination order information.

To achieve at least one of the abovementioned objects, according to an aspect of the present disclosure, an information processing apparatus reflecting one aspect of the present disclosure creates examination order information for capturing a medical image, and includes a hardware processor that
acquires at least one reference image candidate,
selects a reference image from the at least one reference image candidate,
causes a display to display examination order information linked with the selected reference image, and
outputs the reference image in association with the examination order information.

### BRIEF DESCRIPTION OF THE DRAWINGS

The advantages and features provided by one or more embodiments of the present disclosure will become more fully understood from the detailed description given hereinafter and the appended drawings which are given by way of illustration only, and thus are not intended as a definition of the limits of the present disclosure, and wherein:

FIG. 1 is a diagram showing an overall configuration of an ordering system;

FIG. 2 is a flowchart illustrating an examination order information creation process;

FIG. 3 is an image diagram of an examination order information creation screen;

FIG. 4 is a flowchart illustrating the examination order information creation process;

FIG. 5 is an image diagram of a reference image search screen; and

FIG. 6 is a flowchart illustrating the examination order information creation process.

### DETAILED DESCRIPTION

Hereinafter, one or more embodiments of the present disclosure will be described in detail with reference to the drawings. However, the scope of the present disclosure is not limited to the disclosed embodiments or the illustrated examples.

### Ordering System 100

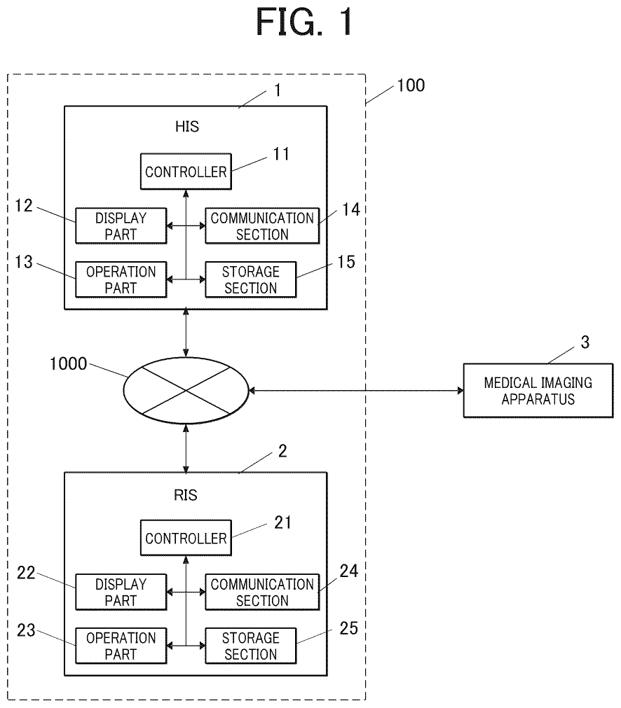

FIG. 1 shows a system configuration of an ordering system 100. The ordering system 100 is a system that executes creation of examination order information for capturing a medical image, transmission of the examination order information, and the like.

As shown in FIG. 1, the ordering system 100 includes an HIS (Hospital Information System) 1 and an RIS (Radiology Information System) 2 which are connected to each other via a network 1000 such as a LAN (Local Area Network) so as to be capable of data communication. Also, a medical imaging apparatus 3 is connected to the ordering system 100 via the network 1000 such as a LAN (Local Area Network) so as to be capable of data communication.

A DICOM (Digital Imaging and Communications in Medicine) standard is applied to the network 1000.

The HIS 1 is an information processing apparatus that is used by a requesting doctor and creates examination order information for capturing a medical image.

The RIS 2 is an information processing apparatus that is used by an examiner, receives the examination order information from the HIS 1, and transmits the examination order information to the medical imaging apparatus 3.

The medical imaging apparatus 3 is an apparatus that captures a medical image.

As the medical imaging apparatus 3, a computed radiography (CR) apparatus, a computed tomography (CT) apparatus, a magnetic resonance imaging (MRI) apparatus, or the like is used.

### HIS 1

Next, the configuration of the HIS 1 will be described with reference to FIG. 1. As illustrated in FIG. 1, the HIS 1 includes a controller 11 (hardware processor), a display part 12 (display), an operation part 13, a communication section 14, and a storage section 15.

The controller 11 includes a central processing unit (CPU), a random access memory (RAM), and the like. The controller 11 reads various programs stored in the storage section 15, loads the programs into the RAM, executes various processes in accordance with the loaded programs, and controls the operation of each component of the HIS 1.

The controller 11 functions as an acquisition section that acquires reference image candidates, and acquires reference image candidates set by default or acquires reference image candidates using machine learning on the basis of part of examination order information.

Each reference image candidate is an image similar to a medical image that the requesting doctor wants to capture, and is a candidate for a reference image that the examiner refers to. The reference image is not limited to a past medical image that has actually been captured, but may be a schematic diagram or the like that imitates a medical image. Further, the reference image may be associated with a related image, such as a medical image related to the reference image or a schematic diagram imitating the medical image.

The controller 11 may acquire reference image candidates stored in advance (set by default) in the storage section 15 described later.

The controller 11 may select and acquire a reference image candidate(s) from one or more reference images stored in the storage section 15, which will be described later, using machine learning, such as artificial intelligence (AI), based on part of examination order information input by the requesting doctor using the operation part 13, which will be described later. Note that the reference image is also provided with an explanation of the reference image (imaging part, age, gender, physique, and the like), and the selection may be performed using information including the explanation. Furthermore, the part of the examination order information includes, for example, attribute information (gender, age, physique, disease name, and the like) of the patient.

The controller 11 functions as a selector that selects a reference image from the reference image candidates.

When the controller 11 receives a signal for selecting a reference image from the requesting doctor using the operation part 13 to be described later, the controller 11 selects the reference image. Furthermore, when there is a related image related to the selected reference image, the controller 11 may also select the related image.

The controller 11 functions as a display controller that causes a display part (display), such as the display part 12, to display the examination order information linked with the selected reference image. The display part is not limited to the display part 12, and may be any of various display devices that is a separate component from the HIS 1 and connected to the HIS 1.

The controller 11 functions as an output section that outputs the reference image in association with the examination order information.

The controller 11 associates the reference image with the examination order information by, for example, attaching the reference image to the examination order information, and outputs the examination order information to the medical imaging apparatus 3. Furthermore, when there is a related image related to the selected reference image, the controller 11 may also output the related image.

The display part 12 includes a monitor such as a liquid crystal display (LCD), and displays various screens (an examination order information creation screen, a reference image search screen, and the like to be described later) in accordance with instructions of display signals input from the controller 11.

The operation part 13 includes a keyboard having cursor keys, number input keys, various function keys, and the like, a pointing device such as a mouse, a touch screen layered on the surface of the display part 12, and/or the like. The operation part 13 is operable by an operator. The operation part 13 outputs various signals based on operations performed by the operator to the controller 11.

The communication section 14 can transmit and receive various signals and various data to and from other apparatuses or the like connected via the network 1000.

The storage section 15 includes a nonvolatile semiconductor memory, hard disk, and/or the like, and stores various programs to be executed by the controller 11, parameters necessary for execution of the programs, various data, and the like.

The storage section 15 stores examination order information linked with a reference image. Note that the examination order information linked with the reference image is not limited to the actually used examination order information, but may be illustratively created examination order information.

### RIS 2

Next, the configuration of the RIS 2 will be described with reference to FIG. 1. As illustrated in FIG. 1, the RIS 2 includes a controller 21, a display part 22, an operation part 23, a communication section 24, and a storage section 25.

The controller 21 includes a central processing unit (CPU), a random access memory (RAM), and the like. The controller 21 reads various programs stored in the storage section 25, develops the programs in the RAM, executes various processes in accordance with the developed programs, and controls the operation of each unit of the RIS 2.

The display part 22 includes a monitor such as a liquid crystal display (LCD) and displays various screens (such as an examination order information creation screen to be described later) in accordance with instructions of display signals input from the controller 21.

The operation part 23 includes a keyboard having cursor keys, number input keys, various function keys, and the like, a pointing device such as a mouse, a touch screen layered on the surface of the display part 22, and/or the like. The operation part 23 is operable by an operator. The operation part 23 outputs various signals based on operations performed by the operator to the controller 21.

The communication section 24 can transmit and receive various signals and various data to and from other apparatuses or the like connected via the network 1000.

The storage section 25 includes a nonvolatile semiconductor memory, hard disk, and/or the like, and stores various programs to be executed by the controller 21, parameters necessary for execution of the programs, various data, and the like.

### Examination Order Information Creation Process Case 1

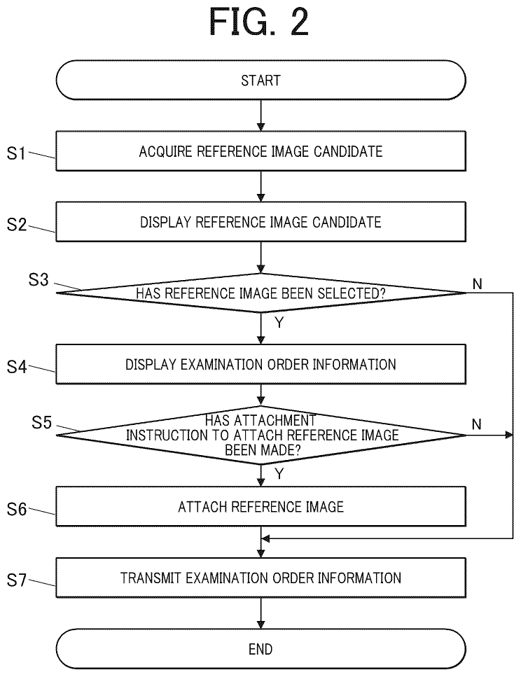

An examination order information creation process will be described with reference to FIG. 2.

The examination order information creation process is a process in which the requesting doctor creates examination order information to be transmitted to the examiner.

In the examination order information creation process shown in FIG. 2, the requesting doctor creates examination order information by selecting a reference image from reference image candidates set by default and editing examination order information linked with the reference image.

First, the controller 11 acquires reference image candidates set by default from the storage section 15 (Step S1; acquiring step).

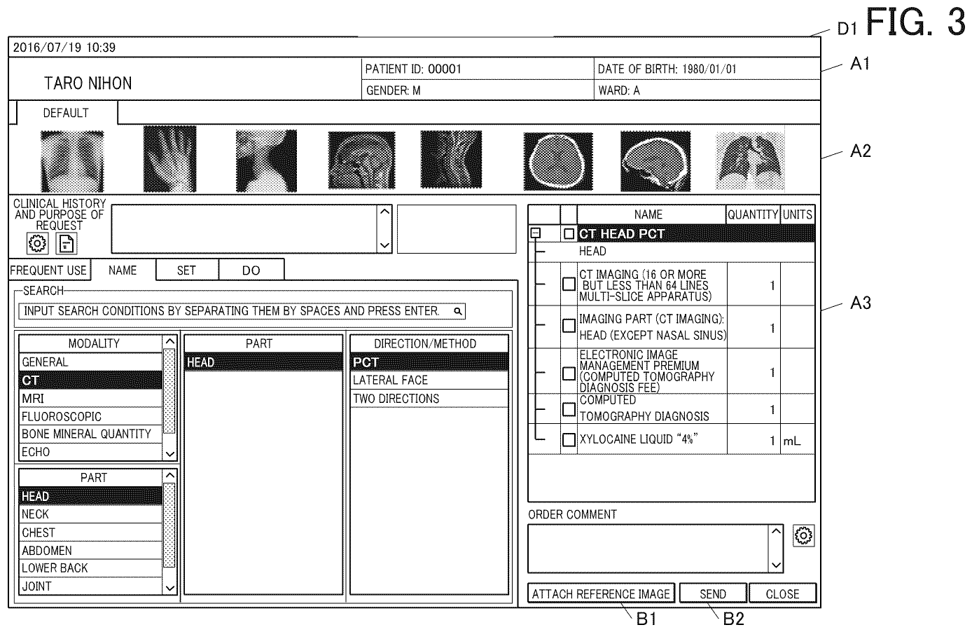

Next, the controller 11 causes the display part 12 to display the reference image candidates (Step S2). To be specific, the controller 11 causes the display part 12 to display an examination order information creation screen D1 illustrated in FIG. 3.

Hereinafter, the examination order information creation screen D1 illustrated in FIG. 3 will be described.

The examination order information creation screen D1 is a screen for the requesting doctor to create examination order information.

The area A1 is an area in which patient information is displayed.

The area A2 is an area in which a reference image is displayed. The requesting doctor selects a reference image from reference image candidates displayed in the area A2 by using the operation part 13.

The area A3 is an area in which examination order information is displayed.

The button B1 is a button for attaching a reference image to examination order information. When the button B1 is pressed, a reference image is attached to examination order information. Note that if there is a related image, the related image may also be attached. The related image to be attached may be selected by the requesting doctor.

The button B2 is a button for transmitting examination order information to the RIS 2.

Next, the controller 11 determines whether the requesting doctor has selected a reference image by using the operation part 13 (Step S3; selecting step). If a reference image is selected (Step S3; YES), the controller 11 advances the examination order information creation process to Step S4. If no reference image is selected (Step S3; NO), the controller 11 advances the examination order information creation process to Step S7.

Next, the controller 11 acquires examination order information linked with the selected reference image from the storage section 15 and causes the display part 12 to display the examination order information (Step S4; display controlling step).

Thus, the requesting doctor can create examination order information by appropriately editing the examination order information displayed on the display part 12 by using the operation part 13, thus saving time and effort to create the examination order information from scratch. Further, by selecting one of reference image candidates displayed by default, examination order information can be displayed, which is a simple operation.

Next, the controller 11 determines whether the requesting doctor has made an attachment instruction to attach the reference image to the examination order information by using the operation part 13 (Step S5). If there is an attachment instruction (Step S5; YES), the controller 11 advances the examination order information creation process to Step S6. If there is no attachment instruction (Step S5; NO), the controller 11 advances the examination order information creation process to Step S7.

Next, the controller 11 attaches the reference image to the examination order information (Step S6).

Note that when there is a related image related to the reference image, the controller 11 may also attach the related image.

Next, the controller 11 transmits the examination order information to the RIS 2 using the communication section 14 (Step S7; outputting step), and ends the examination order information creation process.

### Examination Order Information Creation Process Case 2

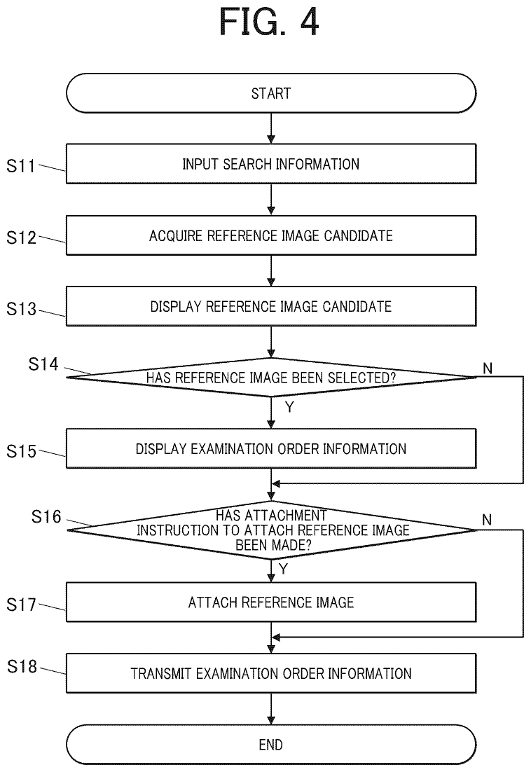

The examination order information creation process will be described with reference to FIG. 4.

In the examination order information creation process shown in FIG. 4, the requesting doctor creates examination order information by inputting part of the examination order information, on the basis of the part of the examination order information, selecting a reference image from among displayed reference image candidates, and editing examination order information linked with the reference image.

First, the controller 11 receives an input of search information (part of examination order information) made by the requesting doctor (Step S11). As described above, the part of examination order information is, for example, attribute information (gender, age, physique, disease name, and the like) of a patient.

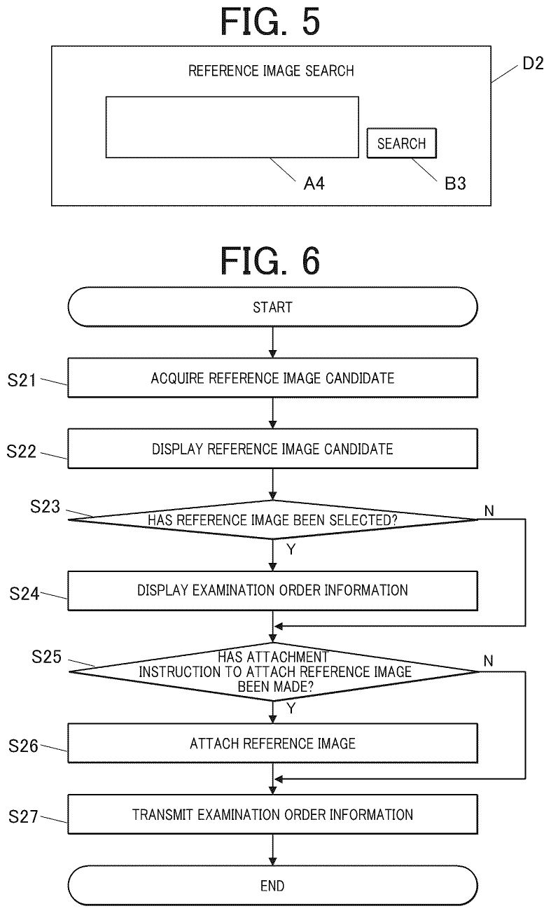

To be specific, the controller 11 causes the display part 12 to display a reference image search screen D2 shown in FIG. 5, and receives an input of search information made by the requesting doctor using the operation part 13.

Hereinafter, the reference image search screen D2 shown in FIG. 5 will be described.

The reference image search screen D2 is a screen for the requesting doctor to search for a reference image similar to a desired medical image.

The area A4 is an area in which the requesting doctor inputs search information for searching for a reference image. For example, the area A4 is a text box, a pull-down, or the like. The area A4 is one text box in the example of FIG. 5, but not limited to the example of FIG. 5, and for example, may be text boxes provided for respective items (imaging part, disease name, or the like). An image may be input into the area A4.

Next, based on the search information, the controller 11 acquires reference image candidates from the storage section 15 using machine learning (Step S12; acquiring step).

Next, the controller 11 causes the display part 12 to display the reference image candidates (Step S13). To be specific, the controller 11 causes the display part 12 to display the examination order information creation screen D1 illustrated in FIG. 3.

Thus, by acquiring and displaying the retrieved reference image candidates, even when the number of reference image candidates has increased, it is possible to display the reference image candidates on the display part in an easily viewable manner. Furthermore, by using machine learning for the search, reference image candidates can be retrieved even if the granularity of the search information varies, and the requesting doctor can easily search for (retrieve) reference image candidates.

The examination order information creation screen D1 in Case 2 is different from the screen in Case 1 only in behavior at the time when the button B1 is pressed.

In Case 2, when the button B1 is pressed, a confirmation dialog (not shown) is displayed to confirm whether a reference image to be attached is a reference image for displaying examination order information linked with or another reference image. although a reference image is attached in either case. If attachment of another reference image is selected on the confirmation dialog, a reference image to be attached is newly selected. Note that similarly to the above, in a case where there is a related image, the related image may also be attached. The related image to be attached may be selected by the requesting doctor.

Next, the controller 11 determines whether the requesting doctor has selected a reference image by using the operation part 13 (Step S14; selecting step). If a reference image is selected (Step S14;YES), the controller 11 advances the examination order information creation process to Step S15. If no reference image is selected (Step S14; NO), the controller 11 advances the examination order information creation process to Step S16.

In Case 2, even if no reference image is selected in Step S14, a reference image to be attached is determined on the confirmation dialog. Therefore, if no reference image is selected in Step S14, the controller 11 advances the examination order information creation process to Step S16, unlike Case 1. That is, in the case where a reference image is attached, a reference image used for displaying examination order information is unconditionally attached in Case 1, but a reference image to be attached is newly selected in Case 2.

As described above, although in Case 1, the determination on the presence or absence of an attachment instruction is not performed if no reference image is selected, in Case 2, the determination on the presence or absence of an attachment instruction is performed even if no reference image is selected. This makes it possible to attach a reference image if the requesting doctor creates examination order information without using examination order information linked with a reference image but wants to attach a reference image for sharing a completed image of the examination order information.

Note that the subsequent processes (Step S15 to Step S18) are the same as those in Step S4 to Step S7 described above, and therefore descriptions thereof will be omitted.

### Examination Order Information Creation Process Case 3

The examination order information creation process will be described with reference to FIG. 6.

In the examination order information creation process shown in FIG. 6, the requesting doctor creates examination order information by selecting a reference image from reference image candidates set by default and editing examination order information linked with the reference image.

First, the controller 11 acquires reference image candidates set by default from the storage section 15 (Step S21; acquiring step).

Next, the controller 11 causes the display part 12 to display the reference image candidates (Step S22). To be specific, the controller 11 causes the display part 12 to display the examination order information creation screen D1 illustrated in FIG. 3.

The examination order information creation screen D1 in Case 3 is different from the screen in Case 1 only in behavior at the time when the button B1 is pressed.

In Case 3, as in Case 2, when the button B1 is pressed, a confirmation dialog (not shown) is displayed to confirm whether a reference image to be attached is a reference image for displaying examination order information linked with or another reference image, although a reference image is attached in either case. If attachment of another reference image is selected on the confirmation dialog, a reference image to be attached is newly selected. Note that similarly to the above, in a case where there is a related image, the related image may also be attached. The related image to be attached may be selected by the requesting doctor.

Next, the controller 11 determines whether the requesting doctor has selected a reference image by using the operation part 13 (Step S23; selecting step). If a reference image is selected (Step S23; YES), the controller 11 advances the examination order information creation process to Step S24. If no reference image is selected (Step S23; NO), the controller 11 advances the examination order information creation process to Step S25.

In Case 3, as in Case 2, even if no reference image is selected in Step S23, a reference image to be attached is determined on the confirmation dialog. Therefore, if no reference image is selected in Step S23, the controller 11 advances the examination order information creation processing to Step S25, unlike Case 1.

As an assumption in a medical site, there is a case where the requesting doctor wants to display and create examination order information based on the last imaging, and therefore the last captured image is used as a reference image for displaying examination order information. However, the following cases are expected: a case where the requesting doctor attaches, as a reference image to be attached, an image that is an image of the same part and/or captured under the same condition but has been subjected to different image processing, such as enhancement processing, because he/she wants to make a predetermined portion in the lung fields to be more visible this time, and a case where the requesting doctor attaches a better image to convey an order to make this bone of a joint more visible.

Note that the subsequent processes (Step S24 to Step S27) are the same as those in Step S4 to Step S7 described above, and therefore descriptions thereof will be omitted.

### Others

In the above, the ordering system 100 uses the HIS 1 and the RIS 2 in accordance with the actual situation of a general medical site, but not limited to this example.

Various information processing terminals or the like may be caused to function as the HIS 1 and the RIS 2 to form the ordering system 100.

Furthermore, in the above, machine learning is performed by the controller 11, but not limited to this example.

A cloud server or the like connected to the HIS 1 may have a function of machine learning.

### Advantageous Effects

As described above, the information processing apparatus (HIS 1) is an information processing apparatus (HIS 1) that creates examination order information for capturing a medical image, and includes an acquisition section (controller 11) that acquires reference image candidate(s), a selector (controller 11) that selects a reference image from the reference image candidates, a display controller (controller 11) that causes a display part to display examination order information linked with the reference image selected by the selector, and an output section (controller 11) that outputs the reference image in association with the examination order information.

Therefore, the requesting doctor can reduce miscommunication with the examiner without taking time and effort in creating examination order information.

An information processing method is an information processing method that is performed by an information processing apparatus that creates examination order information for capturing a medical image, and includes: an acquiring step (Step S1, Step S12) of acquiring reference image candidates; a selecting step (Step S3, Step S14) of selecting a reference image from the reference image candidates; a display controlling step (Step S4, Step S15) of causing a display part to display examination order information linked with the reference image selected in the selecting step; and an outputting step (Step S7, Step S18) of outputting the reference image in association with the examination order information.

Therefore, the requesting doctor can reduce miscommunication with the examiner without taking time and effort in creating examination order information.

The program causes a computer of an information processing apparatus (HIS 1) that creates examination order information for capturing a medical image to function as an acquisition section (controller 11) that acquires reference image candidates, a selector (controller 11) that selects a reference image from the reference image candidates, a display controller (controller 11) that causes a display part to display examination order information linked with the reference image selected by the selector, and an output section (controller 11) that outputs the reference image in association with the examination order information.

Therefore, the requesting doctor can reduce miscommunication with the examiner without taking time and effort in creating examination order information.

Note that those described in the above embodiment are examples of the ordering system and the information processing apparatus according to the present disclosure, and the present disclosure is not limited thereto. The detailed configuration and detailed operation of each component constituting the apparatus can be changed as appropriate without departing from the scope of the present disclosure.

In the above description, as a computer-readable medium storing a program for executing each process, a ROM or a hard disk is used, but the present disclosure is not limited thereto. As the computer-readable medium, a nonvolatile memory, such as a flash memory, and a portable recording medium, such as a CD-ROM, can also be used. In addition, a carrier wave may be applied as a medium that provides data of the program via a communication line.

Although embodiments of the present disclosure have been described and illustrated in detail, the disclosed embodiments are made for purposes of illustration and example only and not limitation. The scope of the present disclosure should be interpreted by terms of the appended claims.

What is claimed is:

1. An information processing apparatus that creates examination order information for capturing a medical image, the information processing apparatus comprising a hardware processor that
acquires at least one reference image candidate,
selects a reference image from the at least one reference image candidate,
causes a display to display examination order information linked with the selected reference image, and
outputs the reference image in association with the examination order information.

2. The information processing apparatus according to claim 1, wherein the hardware processor acquires the reference image candidate set by default.

3. The information processing apparatus according to claim 1, wherein the hardware processor acquires the reference image candidate using machine learning based on a part of the examination order information.

4. The information processing apparatus according to claim 1, wherein the reference image candidate includes a schematic diagram.

5. An information processing method that is performed by an information processing apparatus that creates examination order information for capturing a medical image, the information processing method comprising:
acquiring at least one reference image candidate;
selecting a reference image from the at least one reference image candidate;
causing a display to display examination order information linked with the selected reference image; and
outputting the reference image in association with the examination order information.

6. A non-transitory computer-readable storage medium storing a program causing, of an information processing apparatus that creates examination order information for capturing a medical image, a computer to:
acquire at least one reference image candidate;
select a reference image from the at least one reference image candidate;
cause a display to display examination order information linked with the selected reference image; and
output the reference image in association with the examination order information.
